(19) United States
(12) Patent Application Publication
(10) Pub. No.: US 2025/0049787 A1
(43) Pub. Date: Feb. 13, 2025

(54) TREATMENT FOR ACUTE MYELOID LEUKEMIA OR LYMPHOMA

(71) Applicant: UNIVERSITY HEALTH NETWORK, Toronto (CA)

(72) Inventors: Mark R. Bray, Oakville (CA); Jacqueline M. Mason, Toronto (CA); Xin Wei, Markham (CA); Gordon Duncan, Toronto (CA)

(21) Appl. No.: 18/723,094

(22) PCT Filed: Dec. 21, 2022
(86) PCT No.: PCT/CA2022/051876

§ 371 Date: Jun. 21, 2024

Related U.S. Application Data
(60) Provisional application No. 63/292,481, filed on Dec. 22, 2021.

(51) Int. Cl. A61K 31/496; A61K 31/635; A61K 31/706; A61P 35/00; A61P 35/02

(52) U.S. Cl. CPC A61K 31/496; A61K 31/635; A61K 31/706; A61P 35/00; A61P 35/02

(57) ABSTRACT

The invention is related to a method of treating a subject with acute myeloid leukemia, acute lymphoblastic leukemia, non-Hodgkin's lymphoma, Burkitt lymphoma, or diffuse large B-cell lymphoma by administration of Compound (I), or a pharmaceutically acceptable salt thereof.

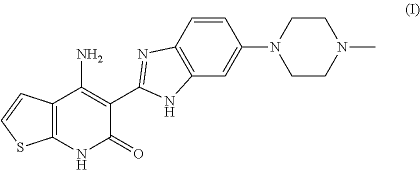 [SMILES: CN1CCN(c2ccc3nc(-c4c(N)c5ccsc5[nH]c4=O)[nH]c3c2)CC1]

### CROSS-REFERENCE TO RELATED APPLICATIONS

This application claims priority to U.S. Provisional Application No. 63/292,481, filed Dec. 22, 2021. The entire contents of the aforementioned application are incorporated herein by reference.

### FIELD OF THE INVENTION

Disclosed herein are methods for treating a subject with different subtypes of acute myeloid leukemia (AML), as well as acute lymphoblastic leukemia (ALL), non-Hodgkin's lymphoma (NHL), Burkitt lymphoma, or diffuse large B-cell lymphoma (DLBCL) with 4-amino-5-(6-(4-methylpiperazin-1-yl)-1H-benzo[d]imidazol-2-yl) thieno[2,3-b]pyridin-6 (7H)-one (Compound (I)) or a pharmaceutically acceptable salt thereof.

### BACKGROUND OF THE INVENTION

Hematopoietic progenitor kinase 1 (HPK1) is a hematopoietic cell-restricted Ste20 serine/threonine kinase. HPK1 kinase activity can be induced by activation signals generated by various different cell surface receptors found in hematopoietic cells upon ligand engagement. Agents which inhibit HPK1 have the potential to treat cancer. A number of potent HPK1 inhibitors are disclosed in U.S. Pat. Nos. 10,501,474 and 11,059,832 (the entire teachings of which are incorporated herein by reference). The structure of one inhibitor disclosed in these patents is shown below as Compound (I).

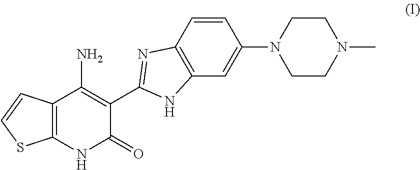 [SMILES: CN1CCN(c2ccc3nc(-c4c(N)c5ccsc5[nH]c4=O)[nH]c3c2)CC1]

Acute myeloid leukemia (AML) is a cancer of the blood and bone marrow. This type of cancer usually gets worse quickly if it is not treated. It is the most common type of acute leukemia in adults.

Development and maintenance of leukemia can be partially attributed to alterations in (anti)-apoptotic gene expression. Genome-wide transcriptome analyses revealed that 89 apoptosis-associated genes were differentially expressed between AML patient CD34⁺ cells and normal bone marrow (NBM) CD34⁺ cells. Among these, transforming growth factor-b activated kinase 1 (TAK1) was strongly upregulated in AML CD34⁺ cells. Genetic down modulation or pharmacologic inhibition of TAK1 activity strongly impaired primary AML cell survival and cobblestone formation in stromal cocultures. TAK1 inhibition was mainly due to blockade of the nuclear factor kB (NF-kB) pathway, as TAK1 inhibition resulted in reduced levels of phospho-IkBa and p65 activity. Overexpression of a constitutively active variant of NF-kB partially rescued TAK1-depleted cells from apoptosis. Importantly, NBM CD34⁺ cells were less sensitive to TAK1 inhibition compared with AML CD34⁺ cells. Knockdown of TAK1 also severely impaired leukemia development in vivo and prolonged overall survival in a humanized xenograft mouse model. It is known that TAK1 is frequently overexpressed in AML CD34⁺ cells, and that TAK1 inhibition efficiently targets leukemic stem/progenitor cells in an NF-kB-dependent manner. However, it cannot be excluded that other pathways besides NF-kB are affected because NF-kB overexpression could only partially rescue the phenotype. See Bosman et al., Blood 2014, 124 (20): 3130-3140.

Internal tandem duplication (ITD) mutations of the FMS-like tyrosine kinase 3 (FLT3) gene have been described in approximately 20% of patients with adult AML: a lower incidence (5%-16.5%) has been reported in childhood leukemia. See Thiede et al., Blood 2002, 99 (12): 4326-4335. In AML patients, the FLT3-ITD mutations were found to be associated with increased leukocyte counts and were frequent in patients lacking other cytogenetic aberrations. More recently, point mutations in codon 835 of the FLT3 gene have been described in approximately 7% of patients with AML. These mutations are located in the activation loop of the second tyrosine kinase domain (TKD) of FLT3 and constitutively activate the protein.

In addition to the juxtamembrane domain mutations, mutations in the tyrosine kinase domain (FLT3-TKD mutations) have been described in AML. FLT3-TKD mutations are small mutations in the activation loop of FLT3, mostly representing point mutations in codon D835 or deletions of codon 1836. They induce constitutive tyrosine phosphorylation leading to activation of the receptor tyrosine kinase and are supposed to represent gain-of-function mutations. See Bacher et al., Blood 2008, 111 (5): 2527-2537.

The mixed-lineage leukemia 1 (MLL1) gene (now renamed Lysine [K]-specific MethylTransferase 2A or KMT2A) on chromosome 11q23 is disrupted in a unique group of acute leukemias. More than 80 different partner genes in these fusions have been described, although the majority of leukemias result from MLL1 fusions with one of about six common partner genes. Approximately 10% of all leukemias harbor MLL1 translocations. See Winters et al., Front. Pediatr. 2017, 5 (4): 1-21. The t(9:11)(p21-22: q23) identifies the second largest group of translocations which involve 11q23 as an acquired abnormality in hemopoietic malignancies. The t(9;11) is most often associated with acute myeloid leukemia (AML), usually those FAB types which have a substantial monocytic element, M4 and M5. At the molecular level, the typical rearrangement is between MLL (also known as ALL1, Htrx, HRX) at 11q23, and a gene known as AF9, MLLT3 or LTG9 at 9p21-22. See Swansbury et al., Leukemia 1998, 12:792-800.

The above described various subtypes of AML, as well as ALL, NHL, Burkitt lymphoma, or DLBCL, are difficult to be treated. The 5-year overall survival rates of the majority of AML cases ranges from 5-15% in older patients to 30% in young adults. See Kuenzi et al., Scientific Reports 2019, 9:606. This lack of improvement in patient survival rates is primarily attributed to the limited efficacy of currently available therapies in AML. Thus, there a need to develop new drugs to treat those diseases.

### SUMMARY OF THE INVENTION

The present disclosure is based on the unexpected discovery that Compound (I) identified above actively inhibits transforming growth factor beta-activated kinase 1 (TAK1) (see Example 1). Moreover, Compound (I) selectively inhibits the growth of FLT3-ITD-expressing AML cells and 32D cell transfectants (see Example 5).

In one aspect, the present disclosure provides a method of treating a subject with acute myeloid leukemia, acute lymphoblastic leukemia, non-Hodgkin's lymphoma, Burkitt lymphoma, or diffuse large B-cell lymphoma, comprising administering an effective amount of Compound (I):

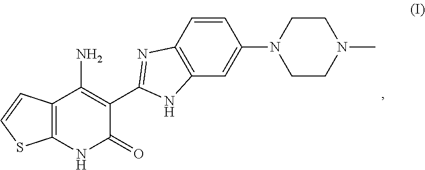 [SMILES: CN1CCN(c2ccc3nc(-c4c(N)c5ccsc5[nH]c4=O)[nH]c3c2)CC1]

or a pharmaceutically acceptable salt thereof, wherein the acute myeloid leukemia
is FLT3 mutated acute myeloid leukemia;
is acute myeloid leukemia with MLL-AF9 translocation;
overexpresses wild-type FLT3;
overexpresses transforming growth factor-b activated kinase 1 (TAK1);
is TAK1 mutated: or
is acute myeloid leukemia with increased TAK1 signaling.

In another aspect, the present disclosure provides the use of Compound (I) or a pharmaceutically acceptable salt thereof for the manufacture of a medicament for treating a subject with different subtypes of acute myeloid leukemia as described above, acute lymphoblastic leukemia, non-Hodgkin's lymphoma, Burkitt lymphoma, or diffuse large B-cell lymphoma.

In another aspect, the present disclosure provides Compound (I) or a pharmaceutically acceptable salt thereof for use in treating a subject with different subtypes of acute myeloid leukemia as described above, acute lymphoblastic leukemia, non-Hodgkin's lymphoma, Burkitt lymphoma, or diffuse large B-cell lymphoma.

### BRIEF DESCRIPTION OF THE DRAWINGS

FIG. 1A shows the inhibitory effect of Compound (I) tartrate against FLT3. FIG. 1B shows the inhibitory effect of Compound (I) tartrate against FLT3-ITD. FIG. 1C shows the inhibitory effect of Compound (I) tartrate against FLT3 (D835Y). FIG. 1D shows the inhibitory effect of Compound (I) tartrate against TAK1/MAP3K7.

FIG. 2A shows the inhibitory effect of Compound (I) tartrate against SLP-76 serine 376 phosphorylation in anti-CD3 antibody stimulated Jurkat E6.1 cells. FIG. 2B shows the inhibitory effect of Compound (I) tartrate against phospho-FLT3 in MV-4-11 cells.

FIG. 3A shows tumor volume change in female CD-1 Nude mice bearing human AML under the treatment with Compound (I) tartrate or Vincristine. FIG. 3B shows body weight change of experimental animals. PO: per os (oral administration): IP: intraperitoneal administration: QD: quaque die (once a day): QW: quaque (once a week); TGI: tumor growth inhibition.

FIG. 4A shows that Compound (I) tartrate treatment increases the expression of the mature 160 kDa form of FLT3-ITD in MV-4-11 cells (FLT3 loss of heterozygosity “LOH”, FLT3-ITD positive cells) in a dose-dependent manner. FIG. 4B shows the increase of cell surface expression of FLT3-ITD in MV-4-11 cells which were treated with 100 nM Compound (I) tartrate or DMSO control for 4 hours at 37° C. and 5% CO₂ and stained with PE-FLT3 (Beckman Coulter, Cat. IM2234U) or an isotype control antibody.

FIG. 5A shows that Compound (I) tartrate treatment (48 and 72 hours at 37° C. and 5% CO₂ and stained with APC-CD45, PECy7-CD38, APC/Fire 750-CD33, PerCP-CD14, PE-FLT3 (Beckman Coulter, Cat. IM2234U), PE/Dazzle 594-CD117 (BioLegend, Cat. #313226) or isotype control antibodies) increases the cell surface expression of FLT3-ITD in a dose-dependent manner in viable CD14 AML blast cells. FIG. 5B shows that the same Compound (I) tartrate treatment increases the cell surface expression of c-KIT (CD117) in viable CD14-AML blast cells.

### DETAILED DESCRIPTION OF THE INVENTION

As used herein, “Compound (I)” refers to a compound having a chemical name 4-amino-5-(6-(4-methylpiperazin-1-yl)-1H-benzo[d]imidazol-2-yl)thieno[2,3-b]pyridin-6(7H)-one, which has the following structure:

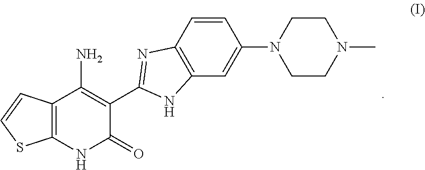 [SMILES: CN1CCN(c2ccc3nc(-c4c(N)c5ccsc5[nH]c4=O)[nH]c3c2)CC1]

Compound (I) was developed as a HPK1 inhibitor and is disclosed in WO2016/205942. The preparation of Compound (I) is described in Example A1 of WO2016/205942, the entire teachings of which are incorporated herein by reference.

The methods disclosed herein can be used to treat certain subtypes of AML which are difficult to be treated in view of specific mutations. AML is the most common type of acute leukemia. It occurs when the bone marrow begins to make blasts, cells that have not yet completely matured. These blasts normally develop into white blood cells. However, in AML, these cells do not develop and are unable to ward off infections.

In AML, the bone marrow may also make abnormal red blood cells and platelets. The number of these abnormal cells increases rapidly, and the abnormal (leukemia) cells begin to crowd out the normal white blood cells, red blood cells and platelets that the body needs.

One of the main features that differentiates AML from the other main forms of leukemia is that it has eight different subtypes, which are based on the type of cell from which the leukemia developed and its degree of maturity. The types of acute myelogenous leukemia include:
Myeloblastic (M0)—on special analysis
Myeloblastic (M1)—without maturation
Myeloblastic (M2)—with maturation
Promyelocytic (M3)
Myelomonocytic (M4)
Monocytic (M5)
Erythroleukemia (M6)
Megakaryocytic (M7)

It was unexpected to find that Compound (I) was effective against FLT3 mutated acute myeloid leukemia. Thus, in one aspect, the present disclosure provides a method of treating a subject with acute myeloid leukemia, wherein the acute myeloid leukemia is FLT3 mutated acute myeloid leukemia. In some embodiments, the FLT3 mutation is FLT3-Internal Tandem Duplication (ITD) mutation and/or FLT3-Tyrosine Kinase Domain (TKD) mutation. Several methods have been developed for identifying FLT3 mutations. These include polymerase chain reaction (PCR)-based approaches, next-generation sequencing (NGS) approaches and multiplex-targeted NGS (i.e., gene panels) approaches. See Daver et al., Leukemia 2019, 33:299-312. In some embodiments, the acute myeloid leukemia has a D835 mutation, e.g., D835Y. In some embodiments, the acute myeloid leukemia is FLT3 mutated acute myeloid leukemia and the AML is selected from AML M1, AML M2, AML M3, AML M4, AML M5, AML M6, and AML M7, for example, AML M5.

The present disclosure provides a method of treating a subject with acute myeloid leukemia, wherein the acute myeloid leukemia is acute myeloid leukemia with MLL-AF9 translocation. In some embodiments, AML is selected from AML M1, AML M2, AML M3, AML M4, AML M5, AML M6, and AML M7, for example, AML M4 or AML M5.

The present disclosure also provides a method of treating a subject with acute myeloid leukemia, wherein the acute myeloid leukemia overexpresses with TAK1, or is TAK1 mutated: or is with increased TAK1 signaling. In some embodiments, AML is selected from AML M1, AML M2, AML M3, AML M4, AML M5, AML M6, and AML M7, for example, AML M4.

It is known that TAK1 is important for the survival of FLT3 mutated AML cells (Shanmugam Clin Cancer Res 2012 18 (2), 360-369), NHL/DLBCL cells (Palakurthi AACR Annual Meeting 2008: Ansell Blood Cancer Journal 2014 4, e183: Wu Cell Biochem Funct 2019 (37) 153-160) and MLL-AF9 leukemic cells (Carretta PLOS ONE 2017 1-18).

Thus, the present disclosure also provides a method of treating a subject with acute myeloid leukemia, wherein the acute myeloid leukemia is FLT3 mutated acute myeloid leukemia (e.g., with FLT3-ITD mutation or TKD mutation), or with MLL-AF9) translocation, and overexpresses TAK1, or is TAK1 mutated, or is with increased TAK1 signaling. In some embodiments, AML to be treated is relapsed or refractory.

In some embodiments, the acute lymphoblastic leukemia to be treated is relapsed or refractory. In some embodiments, ALL to be treated is complex karyotype acute lymphoblastic leukemia.

In some embodiments, the acute lymphoblastic leukemia to be treated is T-cell acute lymphoblastic leukemia. In some embodiments, the acute lymphoblastic leukemia to be treated is B-cell acute lymphoblastic leukemia.

In some embodiments, the non-Hodgkin's lymphoma to be treated is relapsed or refractory. In some embodiments, non-Hodgkin's lymphoma to be treated is complex karyotype non-Hodgkin's lymphoma.

In some embodiments, the Burkitt lymphoma to be treated is relapsed or refractory. In some embodiments, Burkitt lymphoma to be treated is complex karyotype Burkitt lymphoma.

In some embodiments, the diffuse large B-cell lymphoma to be treated is relapsed or refractory. In some embodiments, diffuse large B-cell lymphoma to be treated is complex karyotype diffuse large B-cell lymphoma.

In some embodiments, the diffuse large B-cell lymphoma to be treated is germinal center B cell-like. In some embodiments, the diffuse large B-cell lymphoma to be treated is activated B cell-like.

In some embodiments, the present teachings provide methods of treating a subject with acute myeloid leukemia, acute lymphoblastic leukemia, non-Hodgkin's lymphoma, Burkitt lymphoma, or diffuse large B-cell lymphoma comprising administering to the subject an effective amount of Compound (I) in combination with an additional therapeutic agent. In some embodiments, the additional therapeutic agent is an anti-cancer drug.

An “anti-cancer drug” is a compound, which when administered in an effective amount to a subject with cancer, can achieve, partially or substantially, one or more of the following: arresting the growth, reducing the extent of a cancer (e.g., reducing size of a tumor), inhibiting the growth rate of a cancer, and ameliorating or improving a clinical symptom or indicator associated with a cancer (such as tissue or serum components) or increasing longevity of the subject.

The anti-cancer agent suitable for use in the methods described herein includes an anti-cancer agent that has been approved for the treatment of cancer. In one embodiment, the anti-cancer agent includes, but is not limited to, a targeted antibody, an angiogenesis inhibitor, an alkylating agent, an antimetabolite, a vinca alkaloid, a taxane, a podophyllotoxin, a topoisomerase inhibitor, a hormonal antineoplastic agent and other antineoplastic agents.

In one embodiment, the anti-cancer agents that can be used in methods described herein include, but are not limited to, paclitaxel, docetaxel, 5-fluorouracil, trastuzumab, lapatinib, bevacizumab, letrozole, goserelin, tamoxifen, cetuximab, panitumumab, gemcitabine, capecitabine, irinotecan, oxaliplatin, carboplatin, cisplatin, doxorubicin, epirubicin, cyclophosphamide, methotrexate, vinblastine, vincristine, melphalan, cytarabine, etoposide, daunorubicin, bleomycin, mitomycin and adriamycin and a combination thereof.

In one embodiment, anti-cancer drug is Venetoclax. In one embodiment, anti-cancer drug is 5-Azacytidine. In one embodiment, anti-cancer drug is decitabine.

In the methods disclosed herein, Compound (I) and the additional therapeutic agent are administered concurrently or sequentially.

Included in the present teachings are pharmaceutically acceptable salts of Compound (I). Compound (I) has basic amine groups and therefore can form pharmaceutically acceptable salts with pharmaceutically acceptable acid(s). Suitable pharmaceutically acceptable acid addition salts of Compound (I) include salts of inorganic acids (such as hydrochloric acid, hydrobromic, phosphoric, metaphosphoric, nitric, and sulfuric acids) and of organic acids (such as acetic acid, benzenesulfonic, benzoic, ethanesulfonic, methanesulfonic, succinic, and trifluoroacetic acid acids).

In some embodiments, the present disclosure provides Compound (I) as a tartrate salt. In certain embodiments, the molar ratio between Compound (I) and tartaric acid is 1:1.

Also included in the present disclosure are crystal forms of Compound (I) or the corresponding pharmaceutically acceptable salt. For example, the crystal forms and their preparation methods are disclosed in International Application No. PCT/CA2021/050645, the entire teachings of which are incorporated herein by reference.

The term “an effective amount” means an amount when administered to the subject which results in beneficial or desired results, including clinical results, e.g., inhibits, suppresses or reduces the cancer (e.g., as determined by clinical symptoms or the amount of cancer cells) in a subject as compared to a control. Specifically, “treating a subject with a cancer” includes achieving, partially or substantially, one or more of the following: arresting the growth or spread of a cancer, reducing the extent of a cancer (e.g., reducing size of a tumor or reducing the number of affected sites), inhibiting the growth rate of a cancer, and ameliorating or improving a clinical symptom or indicator associated with a cancer (such as tissue or serum components).

Generally, an effective amount of a compound of the invention varies depending upon various factors, such as the given drug or compound, the pharmaceutical formulation, the route of administration, the type of disease or disorder, the identity of the subject or host being treated, and the like, but can nevertheless be routinely determined by one skilled in the art. An effective amount of a compound of the present invention may be readily determined by one of ordinary skill by routine methods known in the art.

In some embodiments, an effective amount of Compound (I) or a pharmaceutically acceptable salt thereof ranges from about 0.01 to about 1000 mg/kg body weight, alternatively about 0.05 to about 500 mg/kg body weight, alternatively about 0.1 to about 200 mg/kg body weight. The skilled artisan will appreciate that certain factors may influence the dosage required to effectively treat a subject suffering from cancer and these factors include, but are not limited to, the severity of the disease or disorder, previous treatments, the general health and/or age of the subject and other diseases present.

In some embodiments, the methods disclosed therein comprising administering to a subject in need thereof an amount of 1 mg to 200 mg of Compound (I) or a pharmaceutically acceptable salt thereof in an amount equivalent to 1 mg to 200 mg of Compound (I), once a day.

As used herein, the term “treat,” “treating,” or “treatment,” when used in connection with a disorder or condition, includes an effect that results in the improvement of the disorder or condition, e.g., different subtypes of acute myeloid leukemia as described above, acute lymphoblastic leukemia, non-Hodgkin's lymphoma, Burkitt lymphoma, or diffuse large B-cell lymphoma. Improvements in or lessening the severity of any symptom of the disorder or condition can be readily assessed according to standard methods and techniques known in the art.

As used herein, the term “refractory” means a cancer that does not respond to treatment. The cancer may be resistant at the beginning of treatment or it may become resistant during treatment.

Compound (I) and/or pharmaceutically acceptable salts thereof described herein are useful as an active pharmaceutical ingredients (API) as well as materials for preparing pharmaceutical compositions that incorporate one or more pharmaceutically acceptable excipients and is suitable for administration to human subjects.

In some embodiments, the disclosure provides a pharmaceutical composition comprising Compound (I) and/or a pharmaceutically acceptable salt thereof and at least one additional pharmaceutically acceptable excipient. The term “pharmaceutically acceptable excipient.” as used herein, refers to a pharmaceutically acceptable material, composition, and/or vehicle, such as a liquid or solid filler, diluent, excipient, solvent, or encapsulating material. Each excipient must be “pharmaceutically acceptable” in the sense of being compatible with the subject composition and its components and not injurious to the patient. Except insofar as any conventional pharmaceutically acceptable excipient is incompatible with Compound (I) and/or pharmaceutically acceptable salts thereof, such as by producing any undesirable biological effect or otherwise interacting in a deleterious manner with any other component(s) of the pharmaceutically acceptable composition, its use is contemplated to be within the scope of this disclosure.

Some non-limiting examples of materials which may serve as pharmaceutically acceptable excipients include: (1) sugars, such as lactose, glucose, and sucrose: (2) starches, such as corn starch and potato starch: (3) cellulose and its derivatives, such as sodium carboxymethyl cellulose, ethyl cellulose, and cellulose acetate: (4) powdered tragacanth; (5) malt: (6) gelatin: (7) talc: (8) excipients, such as cocoa butter and suppository waxes; (9) oils, such as peanut oil, cottonseed oil, safflower oil, sesame oil, olive oil, corn oil, and soy bean oil: (10) glycols, such as propylene glycol: (11) polyols, such as glycerin, sorbitol, mannitol, and polyethylene glycol: (12) esters, such as ethyl oleate and ethyl laurate; (13) agar: (14) buffering agents, such as magnesium hydroxide and aluminum hydroxide; (15) alginic acid: (16) pyrogen-free water: (17) isotonic saline: (18) Ringer's solution; (19) ethyl alcohol: (20) phosphate buffer solutions; and (21) other non-toxic compatible substances employed in pharmaceutical formulations. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York, the contents of each of which is incorporated by reference herein, also disclose additional non-limiting examples of pharmaceutically acceptable excipients, as well as known techniques for preparing and using the same.

Compound (I) or the corresponding pharmaceutically acceptable salts used in the disclosed methods can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

Compound (I) or the corresponding pharmaceutically acceptable salts used in the disclosed methods can be suitably formulated into pharmaceutical compositions for administration to a subject. The pharmaceutical compositions of the present teachings optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

Typically, for oral therapeutic administration, Compound (I) or the corresponding pharmaceutically acceptable salts used in the disclosed methods may be incorporated with excipient and used in the form of ingestible tablets, buccal tablets, troches, capsules, elixirs, suspensions, syrups, wafers, and the like.

Typically for parenteral administration, solutions of Compound (I) or the corresponding pharmaceutically acceptable salts used in the disclosed methods can generally be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, Compound (I) or the corresponding pharmaceutically acceptable salts used in the disclosed methods for the extemporaneous preparation of sterile injectable solutions or dispersions are appropriate.

For nasal administration, Compound (I) or the corresponding pharmaceutically acceptable salts used in the disclosed methods can be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

For buccal or sublingual administration, Compound (I) or the corresponding pharmaceutically acceptable salts used in the disclosed methods can be formulated with a carrier such as sugar, acacia, tragacanth, or gelatin and glycerine, as tablets, lozenges or pastilles.

For rectal administration, Compound (I) or the corresponding pharmaceutically acceptable salts used in the disclosed methods can be formulated in the form of suppositories containing a conventional suppository base such as cocoa butter.

### EXAMPLES

The following examples are intended to be illustrative and are not meant in any way to limit the scope of the disclosure.

### Example 1. In Vitro Cytotoxicity (IC₅₀) Assays

Kinase IC₅₀ values were determined by Reaction Biology Corp. (Malvern, PA, USA) in the Radiometric HotSpot™ Assay using recombinantly expressed kinase enzymes. Compound (I) was tested in a 10-dose IC₅₀ curve in duplicate. Enzymes were tested at the Km [ATP] for each enzyme using the Reaction Biology binning structure. Kinase reactions were initiated by the addition of protein or peptide substrate and [γ-³³P]-adenosine triphosphate (ATP). Phosphorylated substrates were captured by spotting of the reaction mix on filter membranes, and enzymatic activity was quantified by liquid scintillation counting. Substrates and ATP concentrations are as follows:
FLT3: Peptide substrate, [EAIYAAPFAKKK], 5 μM: ATP 50 μM
FLT3-ITD (internal tandem duplication): Peptide substrate, [EAIYAAPFAKKK], 20 μM: ATP, 30 μM
FLT3 (D835Y): Peptide substrate, [EAIYAAPFAKKK], 5 μM: ATP 20 μM
TAK1: Casein, 1 mg/mL: ATP, 20 μM

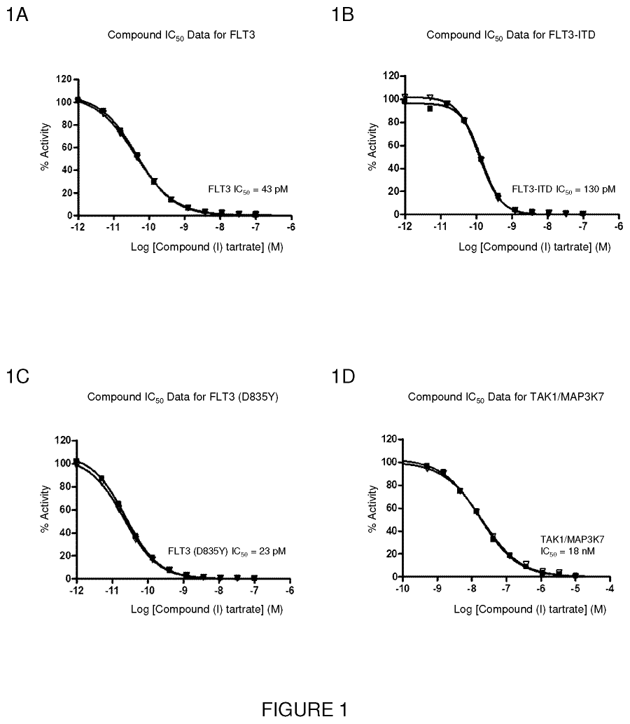

The results are listed in FIGS. 1A-ID. Specifically, Compound (I) tartrate was determined to inhibit FLT3 with an IC₅₀ of 43 pM: FLT3-ITD with an IC₅₀ of 130 pM: FLT3 (D835Y) with an IC₅₀ of 23 pM; and TAK1 with an IC₅₀ of 18 nM.

### Example 2. In Vitro Phosphorylation Assays

### A.

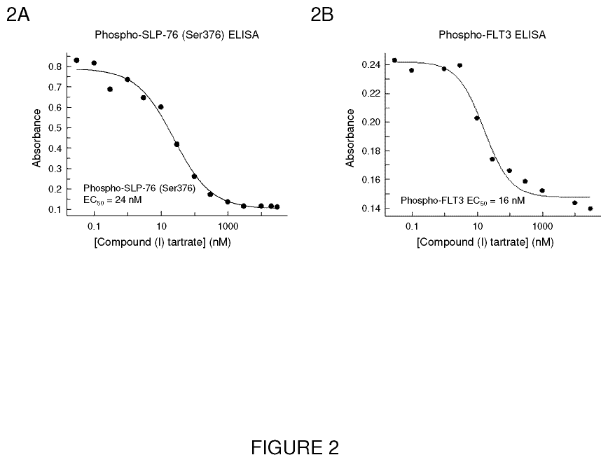

Jurkat E6.1 cells were starved overnight in RPMI-1640 medium (Invitrogen) without fetal bovine serum (FBS). Starved cells (2×10⁶ per sample in 1 mL) were pretreated with Compound (I) tartrate for 3 hours and then stimulated with 5 μg/mL anti-CD3 antibody (clone OKT3) for 15 minutes. Cells were immediately lysed in cell lysis buffer plus protease and phosphatase inhibitors, and then assayed for SLP-76 Ser376 phosphorylation with the PathScan Phospho-SLP-76 (Ser376) Sandwich ELISA Kit according to the kit instructions (Cell Signaling Technology, Inc.). Duplicate data points for each concentration were collected (Molecular Devices M5). Raw absorbance (A=450 nm) values were background subtracted and then individual values were averaged for each Compound (I) tartrate concentration. The cellular EC₅₀ was determined using a simple one-site dose-response curve fitting model (XLfit4, IDBS). Compound (I) tartrate was found to have a phospho-SLP-76 Ser376 EC₅₀ of 24 nM. The results are shown in FIG. 2A.

### B.

MV-4-11 cells were grown in RPMI-1640 medium (Invitrogen) containing 10% FBS. Cells (1×10⁶ per sample in 1 mL) were treated with Compound (I) tartrate for 4 hours and then lysed. Lysates were tested with the PathScan Phospho-FLT3 (panTyr) Sandwich ELISA Kit according to the kit instructions (Cell Signaling Technology, Inc.). Duplicate data points for each concentration were collected (Molecular Devices M5). Raw absorbance (A=450 nm) values were background subtracted and then individual values were averaged for each Compound (I) tartrate concentration. The cellular EC₅₀ was determined using a simple one-site dose-response curve fitting model (XLfit4, IDBS). Compound (I) tartrate was determined to inhibit phospho-FLT3 in MV-4-11 cells with an EC₅₀ of 16 nM. The results are shown in FIG. 2B.

### Example 3. In Vivo Effect of Compound (I) Tartrate on a Xenograft Model of Human AML

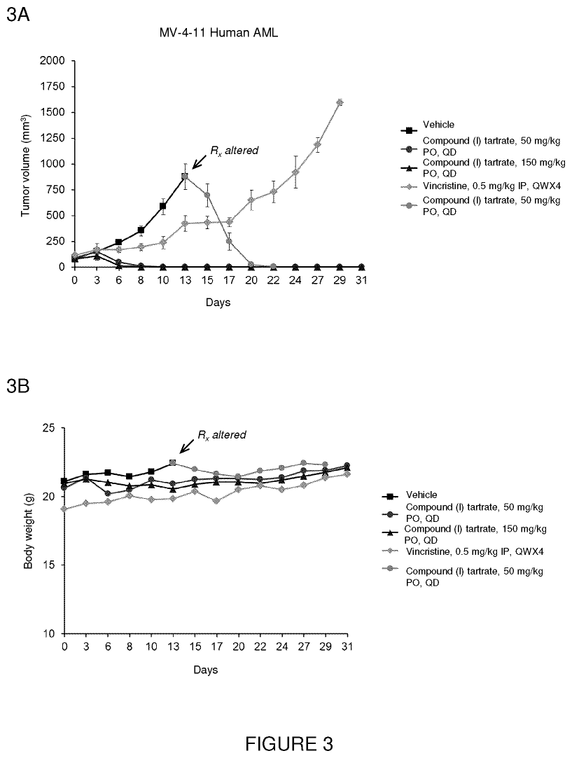

Female 8- to 10-week-old CD-1 Nude mice (Charles River Laboratories) with established MV-4-11 xenografts were treated for 21 days (n=8). MV-4-11 cells is a human AML cell line which endogenously expresses a Fms Related Receptor Tyrosine Kinase 3 (FLT3) internal tandem duplication (ITD) mutation. Animal weights were monitored daily, and tumor volume was measured three times per week. Tumor volume (mm³) was defined as follows, length×width²/2. Percentage tumor growth inhibition (% TGI) was defined as follows, 100×[1−(TV_{f,treated}−TV_{i,treated})/(TV_{f,control}−TV_{i,control})], where TV_{f} is the average tumor volume at the end of study, and TVᵢ is the average tumor volume at the initiation of treatment. In cases where tumor regression occurred, percentage tumor regression was defined as follows, 100×[1−(TV_{f,treated}/TV_{i,treated})]. At the completion of the study, the mice were sacrificed by an anesthetic overdose. The Institutional Animal Care and Use Committee of the University Health Network approved all animal procedures. In mice given orally QD either 50 mg/kg or 150 mg/kg of Compound (I) tartrate tumors were undetectable after 10 days of treatment (FIG. 3A). By contrast, Vincristine had a modest effect on tumor growth (Vincristine 0.5 mg/kg IP, QW×4 vs vehicle, Day 10 TGI=62%, p=0.137). Compound (I) tartrate (50 mg/kg) was also examined in large tumors (≅900 mm³) and complete tumor regression was observed after 7 days of treatment. Data are represented as mean±SEM. p values were calculated using Student's/test.

Compound (I) tartrate was well tolerated as measured by little to no decrease in body weight and normal behavior (FIG. 3B). These results indicate that Compound (I) tartrate can inhibit the growth of human AML cells and may be effective in a clinical setting even in advanced tumors.

### Example 4. Growth Inhibition of Human Leukemia and Lymphoma Cells In Vitro with Compound (I) Tartrate

Cell lines were obtained from American Type Culture Collection (ATCC) and maintained according to the supplier's instructions, and from Dr. Mark Minden (Princess Margaret Cancer Centre, Toronto, Canada). Short tandem repeat (STR) profiling was used to verify authenticity of the cell lines. Peripheral blood from healthy human donors was obtained from the Hematology Malignancy Tissue Bank at the University Health Network. Peripheral blood mononuclear cells (PBMC) were prepared from the blood by density gradient centrifugation using Ficoll-Paque PLUS by manufacturer's instructions (GE Healthcare Life Sciences). For the growth inhibition IC₅₀ assay, cells were seeded at various numbers into 96-well plates (Thermo Fisher Scientific) according to cell growth rate 24 hours before compound overlay and cultured at 37° C. and 5% CO₂. Compound (I) tartrate was prepared as a 10 mM stock solution in 100% DMSO. The stock solution was diluted with RPMI-1640 (Invitrogen) containing 10% FBS (fetal bovine serum) such that the final concentrations ranged from 300 fM to 300 μM. Aliquots (20 μL) from each concentration were overlaid to 180 μL of pre-seeded cells to achieve final concentrations of 30 fM to 30 μM. After 3 days, cell growth in each well was assessed by Alamar Blue assay reagent (Invitrogen). Raw fluorescence (λEM=590 nm) values were adjusted by subtracting the average of the baseline readings from untreated cells assessed one day after cell seeding. Relative cell growth was calculated by comparing to DMSO-treated cells. The concentrations at which cell growth was inhibited by 50% (growth inhibition IC₅₀) were calculated using GraphPad PRISM software (GraphPad Software Inc.). The results are listed in Table 1 below.

TABLE 1

| | | IC₅₀ value for growth |
|---|---|---|
| Cell line | Cell type | inhibition (μM) |
| HL-60 | AML M2 | 1.2 |
| NB4 | AML M3 | 0.94 |
| OCI-AML2 | AML M4 | 1.5 |
| OCI-AML3 | AML M4 | 30 |
| OCI-AML4 | AML M4 | 0.16 |
| OCI-AML5 | AML M4 | 0.27 |
| MOLM-13 | AML M5 | 1.9 |
| MOLM-14 | AML M5 | 0.0047 |
| MV-4-11 | AML M5 | 0.0037 |
| THP-1 | AML M5 | 1.3 |
| U937 | AML M5 | 0.77 |
| OCI-LY3 | NHL, ABC-DLBCL | 0.24 |
| OCI-LY8 | NHL, GCB-DLBCL | 3.6 |
| OCI-LY10 | NHL, ABC-DLBCL | 0.57 |
| OCI-LY18 | NHL, GCB-DLBCL | 0.09 |
| SU-DHL-8 | NHL, GCB-DLBCL | 2.6 |
| DAUDI | NHL, Burkitt's lymphoma | 1.3 |
| RAJI | NHL, Burkitt's lymphoma | 6.6 |
| K562 | CML | 6.0 |
| JURKAT | T-ALL | 3.4 |
| MOLT-4 | T-ALL | 1.6 |
| KOPN-8 | B-ALL | 3.3 |
| NALM-6 | B-ALL | 10 |
| NALM-16 | B-ALL | 1.1 |
| REH | B-ALL | 1.4 |
| RS4; 11 | B-ALL | 1.7 |
| PBMC | NORMAL | 14 |

AML M2: acute myeloblastic leukemia with maturation
AML M3: acute promyelocytic leukemia
AML M4: acute myelomonocytic leukemia
AML M5: acute monocytic leukemia
NHL, Burkitt's lymphoma: Non-Hodgkin lymphoma, Burkitt's lymphoma
NHL, ABC-DLCBL: Non-Hodgkin lymphoma, activated B-cell diffuse large B-cell lymphoma
NHL, GCB-DLCBL: Non-Hodgkin lymphoma, germinal center B-cell diffuse large B-cell lymphoma
CML: chronic myelogenous leukemia
T-ALL: T-cell acute lymphoblastic leukemia
B-ALL: B-cell acute lymphoblastic leukemia
PBMC: peripheral blood mononuclear cell

### Example 5. Growth Inhibition of FLT3-ITD-Expressing Cells In Vitro with Compound (I) Tartrate

Cell lines were obtained from American Type Culture Collection (ATCC) and maintained according to the supplier's instructions, and from Dr. Mark Minden. 32D cell transfectants were obtained from Dr. Mark Minden. Wild-type FLT3-expressing 32D cells were maintained in IMDM media supplemented with 10% heat-inactivated FBS and 50 ng/mL human FLT3 ligand (PeproTech, Inc.). FLT3-ITD-expressing 32D cells were maintained in IMDM media with 10% heat-inactivated FBS without cytokines. The growth inhibition IC₅₀ assay was performed as described in Example 4. Compound (I) tartrate selectively inhibits the growth of FLT3-ITD-expressing AML cells and 32D cell transfectants. The growth inhibition against the FLT3-ITD-expressing 32D cell transfectant is cancelled by the addition of mouse IL-3, indicating the selective inhibitory profile of Compound (I) tartrate against constitutively active FLT3 kinases. The results are listed in Table 2 below.

TABLE 2

| | | | IC₅₀ value for growth |
|---|---|---|---|
| Cell line | Cell type | FLT3 status | inhibition (μM) |
| MV-4-11 | AML M5 | ITD, | 0.0037 |
| | | protein expressed | |
| MOLM-13 | AML M5 | ITD + wild-type, | 1.9 |
| | | protein not expressed | |
| MOLM-14 | AML M5 | ITD + wild-type, | 0.0047 |
| | | protein expressed | |
| HL-60 | AML M2 | wild-type, | 1.2 |
| | | protein expressed | |
| THP-1 | AML M5 | wild-type, | 1.3 |
| | | protein expressed | |

32D Transfectants

| | |
|---|---|
| FLT3-ITD | 0.0063 |
| Wild-type FLT3 (50 ng/ml human FLT3 ligand) | 1.65 |
| FLT3-ITD (20 ng/ml mouse IL-3) | 1.44 |

AML M2: acute myeloblastic leukemia with maturation
AML M5: acute monocytic leukemia

### Example 6. Compound (I) Treatment Increases the Cell Surface Expression of the Glycosylated Mature Form of FLT3-ITD in AML Cells

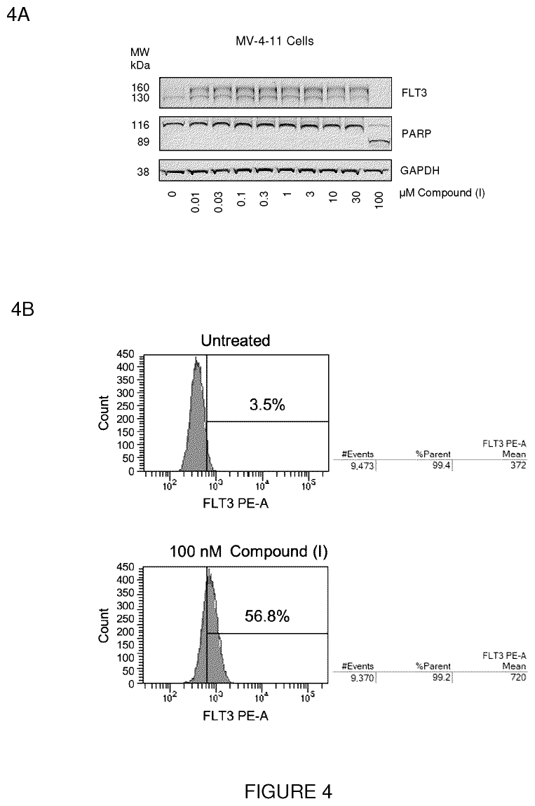

MV-4-11 cells (FLT3 LOH, FLT3-ITD positive cells) were treated with the indicated concentrations of Compound (I) tartrate for 4 hours at 37° C. and 5% CO₂. Lysates were analyzed by immunoblot analysis with antibodies against FLT3 (Cell Signaling Technology, Inc., Cat. #3462), PARP (Cell Signaling Technology, Inc., Cat. #9542) and GAPDH (Millipore, Cat. #MAB374). Protein bands were visualized using a LI-COR Odyssey near infrared imager and representative data are shown. Compound (I) tartrate treatment increases the expression of the mature 160 kDa form of FLT3-ITD in MV-4-11 cells in a dose-dependent manner. Apoptotic cell death as measured by PARP cleavage was induced by Compound (I) tartrate treatment. See FIG. 4A. An increase in FLT3-ITD maturation following Compound (I) tartrate treatment was also observed in MOLM-14 cells, which co-express FLT3-ITD and wild-type FLT3 thus suggesting this mechanism is not restricted to cells with FLT3 LOH (data not shown).

MV-4-11 cells were treated with 100 nM Compound (I) tartrate or DMSO control for 4 hours at 37° C. and 5% CO₂ and stained with PE-FLT3 (Beckman Coulter, Cat. IM2234U) or an isotype control antibody and analyzed on a BD LSRFortessa II flow cytometry analyzer. Data were analyzed using FlowJo software and representative data are shown. As demonstrated in FIG. 4B. Compound (I) tartrate treatment increases the cell surface expression of FLT3-ITD in MV-4-11 cells.

### Example 7. Compound (1) Treatment Increases the Cell Surface Expression of FLT3-ITD and c-KIT (CD117) in Primary Human AML Cells

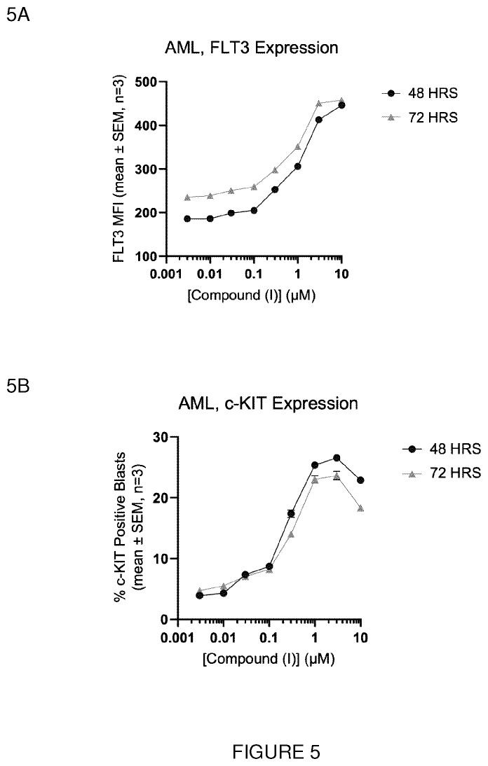

Patient samples were obtained from Dr. Mark Minden (Princess Margaret Cancer Centre, Toronto, Ontario, Canada). FLT3-ITD positive AML cells were treated with the indicated concentrations of Compound (I) for 48 and 72 hours at 37° C. and 5% CO₂ and stained with APC-CD45, PECy7-CD38, APC/Fire 750-CD33, PerCP-CD14, PE-FLT3 (Beckman Coulter, Cat. IM2234U), PE/Dazzle 594-CD117 (BioLegend, Cat. #313226) or isotype control antibodies and analyzed on a BD LSRFortessa II flow cytometry analyzer. Data were analyzed using FlowJo software and representative data are shown. Compound (I) treatment increases the cell surface expression of FLT3 and c-KIT in a dose-dependent manner in viable CD14⁻ AML blast cells. See FIGS. 5A and 5B. Similar results were obtained in additional FLT3-ITD positive primary AML samples (data not shown).

Significance: The majority of FLT3-ITD is sequestered in the endoplasmic reticulum (ER) where constitutive activation of the oncogenic receptor prevents glycosylation, maturation and translocation to the cell surface. Compound (I) inhibits the activity of FLT3-ITD thus allowing for processing to the fully glycosylated mature form and increased cell surface expression. These data demonstrate that Compound (I) increases the cell surface expression of FLT3-ITD and c-KIT (CD117) in AML cells and thus provide a rationale for combining Compound (I) with FLT3-directed and c-KIT-directed immunotherapies (e.g., monoclonal antibodies, bispecific antibodies and CAR T cell therapies) for AML patients. This therapeutic strategy may not only overcome the limitation of FLT3 antigen availability in FLT3-ITD positive AML cells but could also prevent adaptive resistance, which is a frequent problem in single agent treatment.

1. A method of treating a subject with acute myeloid leukemia, comprising administering an effective amount of Compound (I): 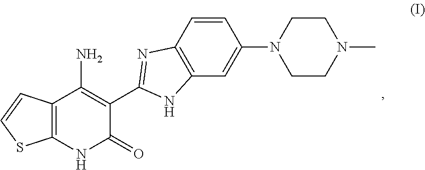 [SMILES: CN1CCN(c2ccc3nc(-c4c(N)c5ccsc5[nH]c4=O)[nH]c3c2)CC1]
or a pharmaceutically acceptable salt thereof, wherein the acute myeloid leukemia
is FLT3 mutated acute myeloid leukemia;
is acute myeloid leukemia with MLL-AF9 translocation;
overexpresses wild-type FLT3;
overexpresses transforming growth factor-b activated kinase 1 (TAK1);
is TAK1 mutated; or
is acute myeloid leukemia with increased TAK1 signaling.

2. The method of claim 1, wherein the acute myeloid leukemia is FLT3 mutated acute myeloid leukemia.

3. The method of claim 2, wherein the FLT3 mutation is FLT3-Internal Tandem Duplication (ITD) mutation.

4. The method of claim 2, wherein the FLT3 mutation is FLT3-Tyrosine Kinase Domain (TKD) mutation.

5. The method of any one of claims 2-4, wherein the acute myeloid leukemia has a D835 mutation, such as D835Y.

6. The method of any one of claims 2-5, wherein the acute myeloid leukemia is selected from AML M1, AML M2, AML M3, AML M4, AML M5, AML M6, and AML M7.

7. The method of claim 6, wherein the acute myeloid leukemia is AML M5.

8. The method of claim 1, wherein the acute myeloid leukemia is acute myeloid leukemia with MLL-AF9 translocation.

9. The method of claim 8, wherein the acute myeloid leukemia is selected from AML M1, AML M2, AML M3, AML M4, AML M5, AML M6, and AML M7.

10. The method of claim 9, wherein the acute myeloid leukemia is AML M4 or AML M5.

11. The method of any one of claims 1-5 and 8, wherein the acute myeloid leukemia overexpresses with TAK1, or is TAK1 mutated; or is with increased TAK1 signaling.

12. The method of claim 11, wherein the acute myeloid leukemia is selected from AML M1, AML M2, AML M3, AML M4, AML M5, AML M6, and AML M7.

13. The method of claim 12, wherein the acute myeloid leukemia is AML M4.

14. The method of any one of claims 1-13, wherein the acute myeloid leukemia is relapsed or refractory.

15. A method of treating a subject with acute lymphoblastic leukemia, non-Hodgkin's lymphoma, Burkitt lymphoma, or diffuse large B-cell lymphoma, comprising administering an effective amount of Compound (I): 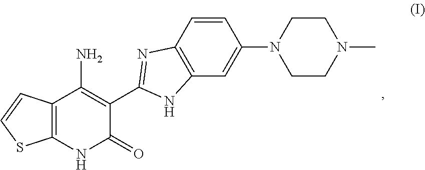 [SMILES: CN1CCN(c2ccc3nc(-c4c(N)c5ccsc5[nH]c4=O)[nH]c3c2)CC1]
or a pharmaceutically acceptable salt thereof.

16. The method of claim 15, wherein the subject has acute lymphoblastic leukemia.

17. The method of claim 16, wherein the acute lymphoblastic leukemia is T-cell acute lymphoblastic leukemia or B-cell acute lymphoblastic leukemia.

18. The method of claim 15, wherein the subject has non-Hodgkin's lymphoma.

19. The method of claim 15, wherein the subject has Burkitt lymphoma.

20. The method of claim 15, wherein the subject has diffuse large B-cell lymphoma.

21. The method of claim 20, wherein the diffuse large B-cell lymphoma is germinal center B cell-like or activated B cell-like.

22. The method of any one of claims 15-21, wherein the acute lymphoblastic leukemia, chronic myeloid leukemia, non-Hodgkin's lymphoma, Burkitt lymphoma, or diffuse large B-cell lymphoma is relapsed or refractory.

23. The method of any one of claims 1-22, wherein Compound (I) is a tartrate salt.

24. The method of claim 23, wherein the molar ratio between Compound (I) and tartaric acid is 1:1.

25. The method of any one of claims 1-24, further comprising co-administering an additional therapeutic agent.

26. The method of claim 25, wherein the additional therapeutic agent is an anti-cancer drug.

27. The method of claim 26, wherein the anti-cancer drug is Venetoclax.

28. The method of claim 26, wherein the anti-cancer drug is 5-Azacytidine.

29. The method of claim 26, wherein the anti-cancer drug is decitabine.

30. The method of any one of claims 25-29, wherein Compound (I) and the additional therapeutic agent are administered concurrently.

31. The method of any one of claims 25-29, wherein Compound (I) and the additional therapeutic agent are administered sequentially.
